(19) United States
(12) Patent Application Publication
(10) Pub. No.: US 2011/0124701 A1
(43) Pub. Date: May 26, 2011

(54) USE OF HDAC INHIBITORS FOR THE TREATMENT OF LYMPHOMAS

(75) Inventors: Peter Wisdom Atadja, Acton, MA (US); Wenlin Shao, Dedham, MA (US)

(73) Assignee: NOVARTIS AG, Basel (CH)

(21) Appl. No.: 12/955,308

(22) Filed: Nov. 29, 2010

Related U.S. Application Data
(63) Continuation of application No. 12/305,245, filed on Dec. 17, 2008, now abandoned.
(60) Provisional application No. 60/806,026, filed on Jun. 28, 2006.

(51) Int. Cl. A61K 31/405; A61P 35/00

(52) U.S. Cl. 514/415

(57) ABSTRACT

The present invention relates to the use of an HDAC inhibitor, especially an HDAC inhibitor of formula (I):

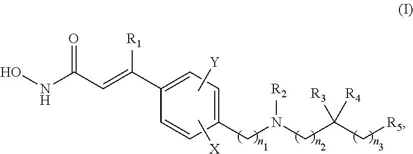

wherein the radicals and symbols have the meanings as defined in the specification, for the preparation of a medicament for the treatment of lymphoproliferative diseases, in particular, cutaneous T-cell lymphomas.

This is a continuation of application Ser. No. 12/305,245 filed on Dec. 17, 2008, which is a National Stage of International Application No. PCT/US2007/14985 filed on Jun. 26, 2007, which claims benefit of U.S. Provisional Application No. 60/806,026 filed Jun. 28, 2006, which in its entirety are herein incorporated by reference.

The present invention relates to the use of an HDAC inhibitor for the preparation of a medicament for the treatment of myeloma; a method of treating a warm-blooded animal, especially a human, having lymphoproliferative diseases, comprising administering to said animal a therapeutically effective amount of an HDAC inhibitor, especially a compound of formula (I) as defined herein; and to a pharmaceutical composition and a commercial package comprising said combination.

The term “lymphoproliferative diseases”, as used herein, relates lymphoproliferative diseases, such as lymphomas especially primary cutaneous T-cell lymphomas (CTCL). Primary CTCL represent a heterogeneous group of non-Hodgkin-lymphomas (NHL) whose etiology. After the group of primary gastrointestinal lymphomas, CTCL together with the primary cutaneous B-cell lymphomas form the second most common group of extra-nodal NHL.

The compounds of formula (I), as defined herein, are histone deacetylase inhibitors (HDAC inhibitors). Reversible acetylation of histones is a major regulator of gene expression that acts by altering accessibility of transcription factors to DNA. In normal cells, histone deacetylase (HDA) and histone acetyltrasferase together control the level of acetylation of histones to maintain a balance. Inhibition of HDA results in the accumulation of hyperacetylated histones, which results in a variety of cellular responses.

Surprisingly, it was now found that HDAC inhibitors, especially the compounds of formula (I), as defined herein, directly inhibit the proliferation of lymphoproliferative diseases, such as CTCL.

Hence, the invention relates to the use of an HDAC inhibitor for the preparation of a medicament for the treatment of lymphoproliferative diseases.

### BRIEF DESCRIPTION OF THE DRAWING

FIG. 1 shows an analysis of a tumor xenograft model of CTCL comparing LBH589 to a vehicle.

### HDAC INHIBITOR COMPOUNDS

HDAC inhibitor compounds of particular interest for use in the inventive combination are hydroxamate compounds described by the formula (I):

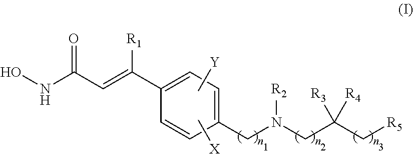

wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-arylalkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₉R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₆alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from 0; NR₁₃; S; and S(O),
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrite; carboxyalkyl; alkylsulfonyl; aminosulfonyl; arylsulfonyl and OR₁₅, such as alkoxy. Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±-3-amin-4-hexenoyl.

Non-aromatic polycycle substituents include bicyclic and tricyclic fused ring systems where each ring can be 4- to 9-membered and each ring can contain zerio, one or more double and/or triple bonds. Suitable examples of non-aromatic polycycles include decalin, octahydroindene, perhydrobenzocycloheptene and perhydrobenzo-M-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, e.g., 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydropyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula

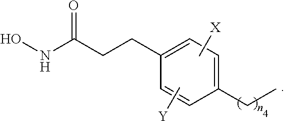

Preferences for each of the substituents include the following:
R₁ is H, halo or a straight-chain C₁-C₄alkyl;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H and C₁-C₆alkyl; or
R₃ and R₄, together with the carbon to which they are bound, represent C═O, C═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₆, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂,
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (Ia):

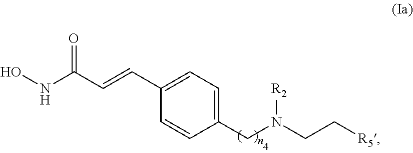

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; and
R₅′ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof.

Another suitable genus of hydroxamate compounds are those of formula (Ia):

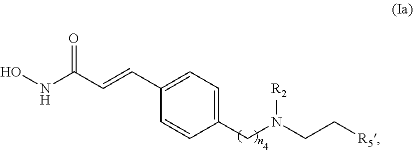

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇; is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl;
   or a pharmaceutically acceptable salt thereof.

Another interesting genus is the compounds of formula (Ib):

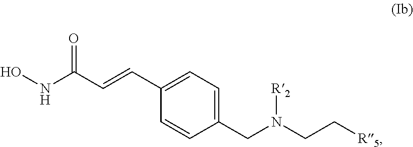

wherein
R₂ is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R₅″; is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (Ic):

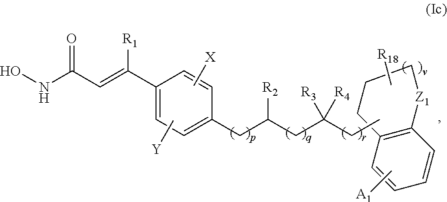

wherein
the ring containing Z₁ is aromatic or non-aromatic, which non-aromatic rings are saturated or unsaturated,
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H; C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
R₁₉ is selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0; or
q is 0 and r is 1-5;
   or a pharmaceutically acceptable salt thereof. The other variable substituents are as defined above.

Especially useful compounds of formula (Ic), are those wherein R₂ is H, or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id):

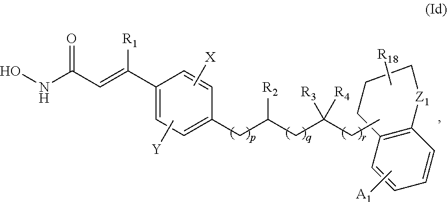

wherein
Z₁ is O, S or N—R₂₀;
R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl;
R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
R₁₉ is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0; or
q is 0 and r is 1-5;
   or a pharmaceutically acceptable salt thereof. The other variable substituents are as defined above.

Especially useful compounds of formula (Id), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie):

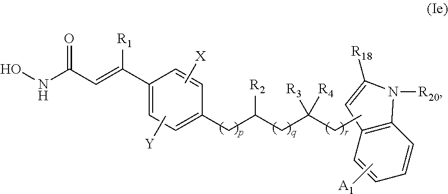

or a pharmaceutically acceptable salt thereof. The variable substituents are as defined above.

Especially useful compounds of formula (Ie), are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (Ie), are those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further relates to the compounds of the formula (If):

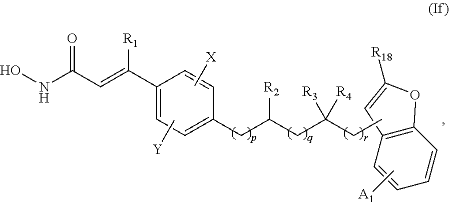

or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, e.g., metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577 published Mar. 21, 2002 which is incorporated herein by reference in its entirety. Two preferred compounds within the scope of WO 02/22577 are:

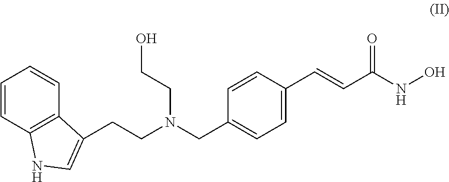

N-hydroxy-3-[4-[(2-hydroxyethyl){2-(1H-indol-3-yl)ethyl]-amino]methyl]phenyl]-2E-2-propenamide, or a pharmaceutically acceptable salt thereof and

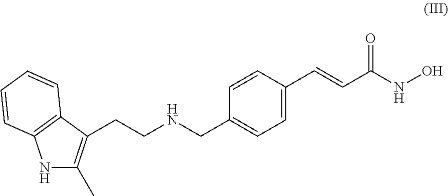

N-hydroxy-3-[4-[[[2-(2-methyl-1H-indol-3-yl)-ethyl]-amino]methyl]phenyl]-2E-2-propenamide, or a pharmaceutically acceptable salt thereof.

The present invention pertains in particular to the use of HDAC inhibitors for the preparation of a medicament for the treatment of myeloma, which is resistant to conventional chemotherapy.

An HDAC inhibitor as used for the present invention displays in the assay described above preferably an IC₅₀ value between 50 and 2500 nM, more preferably between 250 and 2000 nM, and most preferably between 500 and 1250 nM.

Furthermore, the invention relates to a method of treating lymphoproliferative diseases, especially CTCL, comprising administering a therapeutically effective amount of an HDAC inhibitor to a warm-blooded animal, in particular a human, in need thereof, preferably a therapeutically effective amount of a compound of formula (I), as defined above, or the salt of such compound having at least one salt-forming group, to a warm-blooded animal, preferably a human, in need thereof.

Throughout the present specification and claims lymphoproliferative diseases means preferably CTCL.

The term “treatment”, as used herein, comprises the treatment of patients having lymphoproliferative diseases or being in a pre-stage of said disease which effects the delay of progression of the disease in said patients.

The present invention provides a method of treating lymphoproliferative diseases comprising administering a an HDAC inhibitor in an amount which is therapeutically effective against lymphoproliferative diseases to a warm-blooded animal in need thereof.

The person skilled in the pertinent art is fully enabled to select relevant test models to prove the hereinbefore and hereinafter mentioned beneficial effects on lymphoproliferative diseases of a compound inhibiting the HDAC activity. The pharmacological activity of a compound inhibiting the HDAC activity may, e.g., be demonstrated in a suitable clinical study or by means of the Examples described below. Suitable clinical studies are, e.g., open-label non-randomized, dose escalation studies in patients with advanced myeloma.

The present invention also provides the use of a compound of formula (I), as defined herein, and the use of a COMBINATION OF THE INVENTION for the preparation of a medicament for the treatment of lymphoproliferative diseases.

### EXAMPLES

### Clinical

Adult patients with histologically-confirmed, advanced solid tumors or non-Hodgkin's lymphoma including CTCL whose disease has progressed despite standard therapy or for whom no standard therapy exists, will be enrolled onto arms 1-3.

Patients with advanced-stage CTCL were entered into a Phase I study. All patients had progressed following prior systemic therapy. Patients were entered into the DLT, dose limiting therapy, dose level 30 mg M,W,F cohort (n=1) or the subsequent MTD, maximum tolerated dose, dose level 20 mg M,W,F weekly (n=8). Compound (III) was continued until disease progression or unacceptable toxicity. The first three patients had 3 mm punch biopsies from CTCL-involved skin lesions performed at 0, 4, 8 and 24 hours after administration, which were subjected to gene expression profiling. Microarray analysis was performed using the Affymetrix U133 plus 2.0 GeneChip that has 47,000 probesets and interrogates 38,500 genes.

Results: Nine (9) patients with CTCL have been entered to date. Of the 9 patients evaluable for response, 2 attained a complete response (CR), 2 attained a partial response (PR), 1 achieved stable disease (SD) with ongoing improvement, and 4 progressed on treatment (PD). Of particular interest, 2 patients who were initially SD required discontinuation because of toxicities (Grade III diarrhea at week 4, Grade II fatigue at week 12). Both had ongoing improvement in their disease achieving a CR and PR, respectively 3 months later. Of the 4 responding patients, one with a CR (discontinued after 10 doses due to Grade III diarrhea) progressed at 8 m. Microarray data on the first 3 patients (2 CR and 1 PD) demonstrated distinct gene expression response profiles between the 3 patients. Surprisingly, the patient with PD showed the greatest transcriptional response with more than 16,000 genes activated or repressed over the 24-hour time course. Of these responsive genes, close to 60% were activated while 40% were repressed. In contrast, less than 1,000 genes showed a 2-fold change in expression in the 2 patients with a CR with greater than 85% of the genes being repressed.

### Preclinical—Sensitivity of CTCL Cell Lines to Compound III (LBH589) Induced Antiproliferative Activity and Cell Death

Cell lines derived from human CTCL (mycosis fungoides), HUT78, HUT102, HH and MJ were treated with Compound III (LBH589) to assess their sensitivity to the drug. Cells were generally maintained in artificial media, such as Dubelco Modified Eagle Medium (DMEM) or RPMI and supplemented with various levels up to 15% fetal bovine serum. The antibiotics penicillin 100 units/mL) and streptomycin (100 μg/mL) were added to prevent bacterial contamination and maintained at 37° C. and 5% CO₂ environment in a sterile incubator.

### Monolayer Growth Inhibition Assay

The MTT is a colorimetric assay to determine the cell proliferation rate. The yellow tetrazolium MTT (3-(4,5-dimethylthiazolyl-2)-2,5-diphenyltetrazolium bromide) is reduced by metabolically active cells, in part by the action of dehydrogenase enzymes, to generate reducing equivalents, such as NADH and NADPH. The resulting intracellular purple formazan can be solubilized and quantified by spectrophotometric means. The signals produced is directly proportional to the cell numbers. Describing the MTT assay in detail, experiments were done using 6-point or 9-point drug titrations in multi-well tissue culture dishes, with outer rows left empty. Cells were suspended in complete media at densities of between 10³ and 10⁴ cell/mL, respectively, and added per well. The appropriate medium (200 μL) was then added. Twenty-four hours later, 10 μL of MTS solution, were added to one plates to determine the activity at the time of compound addition (T₀). This plate was incubated at 37° C. for 4 hours and the optical density was measured on a Molecular Devices Thermomax at 490 nm using the Softmax program. The T₀ plate served as a reference for initial activity at the beginning of the experiment.

Compound addition began 24 hours after seeding, the same time as the T₀ determination. Serial dilutions at 4-fold, 2-fold, 1-fold, 0.5-fold, 0.25-fold and 0.125-fold of previously determined IC₅₀ values of each compound were made in a 96-deep well plate with the highest concentrations on the edge of the plate. Each of the six dilutions were added in triplicate and complete medium was added to the empty outer rows without cells. The compounds were added to the plates singly or in combination with Compound III (LBH589). The plates were incubated at 37° C. for 72 hours from seeding. The MTS solution was added (as for the T₀ plate) and read four hours later. In order to analyze the data, the average value of media alone (background) was subtracted from each experimental well and the triplicate values were averaged for each compound dilution. The following formulas were used to calculate percent growth.

If X>T₀, % Growth=100×((X−T₀)/(GC−T₀))

If X<T₀, % Growth=100×(X−T₀)/T₀)

T₀=average value of T₀ minus background
GC=average value of untreated cells (in triplicate) minus background
X=average value of compound treated cells (in triplicate) minus background

IC₅₀ the concentration of LBH589 required to inhibit cell growth by 50% and LD₅₀s the concentration required to reduce cell number (kill cells) to 50% the original innoculum were determined. The “% Growth” was plotted against compound concentration and used to calculate IC₅₀s and LD₅₀s, employing the user-defined spline function in Microsoft Excel.

Table 1 shows the antiproliferative effect (IC₅₀) and induction of cell death (LD₅₀) of Compound III (LBH589) in the CTCL cell lines. All four cell lines showed extreme subnanomolar sensitivity (IC₅₀) to the drug, however, only HUT78 and HH were sensitive to LBH589 induced cell death (low nanomolar LD₅₀). It is to be noted that the two cell lines MJ and HUT102 which were insensitive to LBH589 induced death have HTLV infection and this may contribute to their relative insensitivity.

TABLE 1
Anti-Proliferative Effects of Compound III (LBH589) in CTCL Cell Lines

| | Compound III (LBH589) | |
|---|---|---|
| Cell lines | IC₅₀ [μM] | LD₅₀ [μM] |
| HuT78 | 0.0002 | 0.003 |
| HH | 0.0007 | 0.0025 |
| MJ | 0.0003 | >10 |
| HuT102 | 0.0004 | >10 |

### Compound III (LBH589) Induces Regression of CTCL Tumor Xenograft in Mice In Vivo

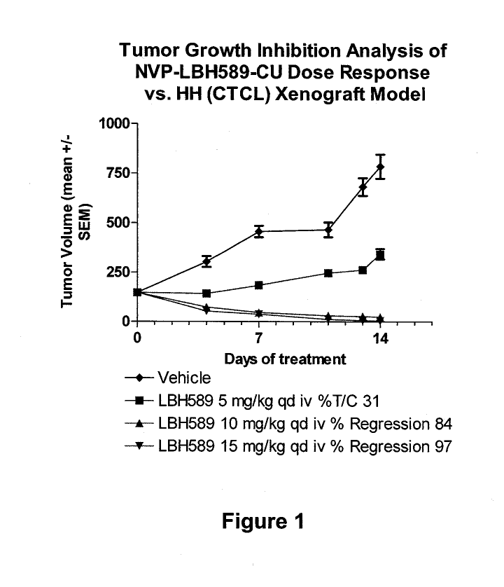

Additionally, Compound III (LBH589) induced the regression of the HH CTCL mouse tumor xenografts in vivo. Mice were implanted with the HH CTCL cell lines and after tumors have grown to 150 mm³, the mice were separated into four groups each containing eight mice. The groups of mice were dosed intravenously with vehicle, 5 mg/kg, 10 mg/kg, or 15 mg/kg daily. The growth of the tumors were followed over 2 weeks and as shown in FIG. 1, 5 mg/kg daily iv dosing of LBH589 induced tumor growth inhibition and both the 10 mg/kg and 15 mg/kg doses produced almost complete tumor regression after 2 weeks of daily dosing.

What is claimed:

1. A method of treating lymphoproliferative diseases comprising administering a therapeutically effective amount of an HDAC inhibitor to a warm-blooded animal in need thereof

2. A method according to claim 1, comprising administering a therapeutically effective amount of a compound of formula (I): 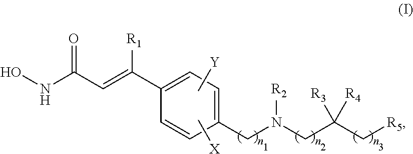
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-arylalkyl-; and —(CH₂)ₙR₇;
R₃ and R₄ are the same or different and, independently, H; C₁-C₆alkyl; acyl; or acylamino; or
R₃ and R₄, together with the carbon to which they are bound, represent C═O, C═S or C═NR₈; or
R₂, together with the nitrogen to which it is bound, and R₃, together with the carbon to which it is bound, can form a C₄-C₉heterocycloalkyl; a heteroaryl; a polyheteroaryl; a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₉; SR₁₅; S(O)R₁₆; SO₂R₁₇; NR₁₃R₁₄; and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H; C₁-C₄alkyl; and —C(O)-alkyl;
R₁₂ is selected from H; C₁-C₉alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl; or
R₁₃ and R₁₄, together with the nitrogen to which they are bound, are C₄-C₉heterocycloalkyl; heteroaryl; polyheteroaryl; non-aromatic polyheterocycle; or mixed aryl and non-aryl polyheterocycle;
R₁₅ is selected from H; C₁-C₆alkyl; C₄-C₉cycloalkyl; C₄-C₉heterocycloalkyl; aryl; heteroaryl; arylalkyl; heteroarylalkyl; and (CH₂)ₘZR₁₂;
R₁₆ is selected from C₁-C₉alkyl; C₄-C₉cycloalkyl; C₄-C₉heterocycloalkyl; aryl; heteroaryl; polyheteroaryl; arylalkyl; heteroarylalkyl; and (CH₂)ₘZR₁₂;
R₁₇ is selected from C₁-C₆alkyl; C₄-C₉cycloalkyl; C₄-C₉heterocycloalkyl; aryl; aromatic polycycles; heteroaryl; arylalkyl; heteroarylalkyl; polyheteroaryl and NR₁₃R₁₄;
m is an integer selected from 0-6; and
Z is selected from O; NR₁₃; S; and S(O),
or a pharmaceutically acceptable salt thereof to a warm-blooded animal in need thereof.

3. A method according to claim 1, wherein the lymphoproliferative diseases is cutaneous T-cell lymphomas.
